(19) United States
(12) Patent Application Publication
(10) Pub. No.: US 2011/0014221 A1
(43) Pub. Date: Jan. 20, 2011

(54) HIV COMBINATION VACCINE AND PRIME BOOST

(75) Inventors: Chil-Yong Kang, London (CA); Chad Michalski, Rockville, MD (US)

Correspondence Address: Eduardo Krupnik, Ontario AgriCentre, 100 Stone Road West, St 301, Canada, ON, N1G 5L3, CA

(73) Assignee: The University of Western Ontario

(21) Appl. No.: 12/522,848

(22) PCT Filed: Jan. 11, 2008
(86) PCT No.: PCT/IB08/00668

§ 371 Date: Feb. 23, 2010

Related U.S. Application Data
(60) Provisional application No. 60/880,103, filed on Jan. 12, 2007.

(51) Int. Cl. A61K 39/21; A61K 39/12

(52) U.S. Cl. 424/186.1; 424/204.1; 424/208.1

(57) ABSTRACT

Provided is a novel, combination prime-boost vaccine against HIV/AIDS that induces long-lasting humoral, cell-mediated and mucosal immune responses against HIV.

### CROSS-REFERENCE TO RELATED APPLICATIONS

This application claims priority to U.S. Provisional Application No. 60/880,103, which was filed on Jan. 12, 2007, the contents of which are hereby incorporated by reference in their entirety.

### BACKGROUND

At the end of 2004, the joint United Nations program on HIV/AIDS (UNAIDS) and the World Health Organization (WHO) released the UNAIDS/WHO global summary of the HIV and AIDS epidemic. In it, they estimate that in the year 2004 alone, over 3 million people worldwide died due to AIDS and despite prevention efforts, a further 5 million became newly infected. This brings the number of individuals currently living with HIV infection to a staggering 40 million. Tragically, treatment options are limited, often economically prohibitive, and ultimately do not represent a cure. Discovering a new means of controlling this devastating disease is imperative and is believed to lie in the development of a safe, effective HIV vaccine.

Despite more than 20 years of research and development, scientists have yet to develop a safe and effective vaccine against HIV. Although we have seen some success in the treatment of HIV infection with the advent of highly active anti-retroviral therapy (HAART), which is capable of temporarily reducing viral loads, this approach suffers from numerous problems including toxicity, prohibitive costs in regions beyond the major economically developed nations, and a continual increase in the number of drug-resistant viral strains. Largely because of these problems, attention has once again shifted back to vaccine development as the best means of controlling HIV infection.

Prevention of HIV infection and AIDS became theoretically achievable with the demonstration that rhesus macaque monkeys immunized with a whole inactivated simian immunodeficiency virus (SIV) or SIV-infected cells were protected against lethal SIV challenge. Since then, numerous methods have been employed to develop a human vaccine against HIV/AIDS. These include inactivated viruses, virus-like particles expressing HIV-1 antigens, live recombinant virus vectors expressing HIV-1 antigens, envelope based subunit vaccines, genetic vaccination using DNA containing HIV-1 gene sequences, multivalent peptide vaccines, and still others. There are inherent advantages and disadvantages associated with all of these approaches, but to date, no approach has proven successful.

Vaccine development against HIV/AIDS continues to struggle with 3 major questions. First, what antigen(s) of HIV are necessary to present to the immune system in order to develop a protective response? Second, what is the most effective method of presenting these antigens to the immune system? And finally, what components of the human immune response confer protection against HIV infection (cellular, humoral or mucosal immunity, or some combination of the three)? A potential vaccine must address all these questions in order to be successful.

### SUMMARY

Provided is a novel, combination prime-boost vaccine against HIV/AIDS that induces long-lasting humoral, cell-mediated and mucosal immune responses against HIV.

In part, the vaccine comprises using a genetically modified HIV, whole-killed virus as the prime injection. This provides maximum stimulation with the native viral surface structures. The priming vaccine is constructed using a rapidly-replicating, avirulent HIV-1 wherein the natural Env glycoprotein signal sequence is replaced with a more efficient and non-cytotoxic one, and wherein a portion of the nef gene is deleted. This genetically altered virus (which can be constructed from not only one, but multiple sub-types of HIV-1), may be produced in large quantities, inactivated and used as a killed, whole-virus vaccine to induce a strong humoral or antibody-mediated immune response. A killed, whole-virus vaccine has the important advantages of expressing virtually all viral proteins to the host immune system, as well as presenting them in their natural, mature conformations.

The vaccine further comprises the use of recombinant adenoviruses delivering a gag-HIV epitope fusion protein forming virus-like-particles as boost immunization modalities. These replication-incompetent recombinant adenovirus (rAd) vectors carrying the HIV gag gene fused with both neutralizing epitope and cytotoxic T-cell epitope regions may be constructed from all major HIV-1 subtypes. These vectors may be produced in a permissive helper cell line which supports their replication, and then they are administered together, where they will be able to infect, but not replicate within host cells and will instead produce virus-like particles which contain the HIV target antigens for presentation to the immune system. Thus, the replication-defective rAd will be used as a boost vaccine and will be capable of inducing not only humoral and cell-mediated immunity, but potentially mucosal immunity as well.

Taken together, we believe that this combination approach represents a powerful HIV/AIDS vaccination method.

Compositions and kits for the practice of the methods are also described herein.

These embodiments of the present invention, other embodiments, and their features and characteristics will be apparent from the description, drawings, and claims that follow. To gain a full appreciation of the scope of the present invention, it will be further recognized that various aspects of the present invention can be combined to make desirable embodiments of the invention.

### BRIEF DESCRIPTION OF THE DRAWINGS

FIG. 1. Construction of combination nef-deleted, EnvNSS replacement mutants. For each strain of HIV-1 being studied, a targeted deletion of the nef gene has been introduced and the natural signal sequence of HIV-1 Env replaced with that of honeybee melittin (A). However, due to overlap between the N-terminal coding region of env and the C-terminal coding region of vpu (A, inset), this results in disruption of the vpu gene as well. Four distinct strains of HIV-1 have been selected for this study based on variation in subtype specificity, cellular tropism, primary versus tissue culture adapted virus, signal sequence length, and the number of positively charged amino acid residues present in the EnvNSS. Most notably, the number of positively charged amino acids present in the EnvNSS has been shown to be critical for efficient Env glycoprotein biosynthesis. In panel B, the phenotypic properties of the selected strains are described briefly, while in C, the Env signal peptides of the respective viruses are shown with positively charged amino acid residues underlined, and the putative signal sequence cleavage site indicated by double bars (//).

FIG. 2. Replication and infectivity of HIV-1_{NL4-3} mutants in A3.01 and H9 cells. Following transfection of proviral DNA, cells were split every 2 days and samples of the culture supernatant collected and analyzed by p24 ELISA in order to monitor viral replication. To assess the infectivity of virus particles being produced, samples were further analyzed by MAGI assay, and the results standardized to represent the number of infectious viral particles present per ng of p24 protein. As shown in this figure the genetically modified combination nef-deleted EnvNSS replacement mutant (NL4-3 T) replicates more rapidly, and to the same or higher titre than wild-type virus (NL4-3 W) in both A3.01 (A) and H9 (B) cells. This occurs despite the wild-type virus being approximately 10-fold (B, inset) to 50-fold (A, inset) more infectious than the NL4-3 T mutant.

FIG. 3. Schematic representation of replication-defective rAd vectors. Replication-defective rAd vectors were generated by cloning in a fusion protein, consisting of a truncated form of the HIV-1 or HIV-2 gag gene fused to a series of neutralizing or T-cell epitopes, into the deleted E1A region of the Ad5 backbone vector. The figure shows each of the 5 rAd vectors (rAd1-5) and the name and amino acid sequence of each of the inserted epitopes. rAd1-3 contain neutralizing epitopes (fused to HIV-2 gag), while rAd4 and 5 contain T-cell epitopes (fused to HIV-1 gag).

FIG. 4. Animal selection and schedule. Eighteen male rhesus macaques were selected for this study and housed at the California National Primate Research Center in Davis, Calif. The individual animal identification number for each subject is shown, as well as the age of the animal at the time the study was initiated. Animals were divided into three groups; Group1, Group2 and Control. The vaccination schedule for each group, including timepoints and immunogen are indicated (AT-2: immunization with AT-2 inactivated whole-killed virus antigen with CpG adjuvant, rAd: immunization with rAd antigen with CpG adjuvant). Each group of animals was subdivided into two further groups based on date of challenge (*this was necessary to accommodate animal 33226, whose vaccination was delayed for a temporary health concern which is believed to be unrelated to the vaccination protocol). Twelve of eighteen animals challenged at week 33 were designated subgroup WOV01, while the remaining six animals challenged at week 39 were designated subgroup WOV02. Viral challenge consisted of a combination of SHIV 89.6 and SHIV SF162p4 viruses administered intravenously. Samples were harvested as indicated in FIG. 5, and necropsies performed on each animal at the dates indicated in that figure.

FIG. 5. Animal body weight measurement. Animals were periodically weighed and examined both pre- and post-vaccination as well as pre- and post-challenge to assess their general health and well-being. All animals tolerated the vaccination protocol well with no measurable loss in weight or negative side-effects (vaccination dates are indicated by yellow arrows). As well, although some animals showed a slight fluctuation in body weight post-challenge (challenge dates are indicated by red arrows), all remained relatively healthy and were monitored for several months until necropsy.

FIG. 6. CD4:CD8 T-cell ratio. The number of CD4+ and CD8+ T-cells per μl of blood sampled were measured by flow cytometry and the CD4:CD8 ratio calculated. Healthy animals normally exhibit a CD4:CD8 ratio >1 (indicated by the dotted line). Animals in all groups, including the unvaccinated control maintained a consistent CD4:CD8 ratio both pre- and post-challenge. Vaccination dates are indicated by light gray arrows and challenge dates are indicated by the dark gray arrows.

FIG. 7. T-cell proliferation assay. Lymphocyte proliferation assays were performed to assess HIV-specific CD4+ T-cell responses. Cells were stimulated with AT-2 inactivated HIV and cell proliferation measured by incorporation of a radio-labelled substrate. A stimulation index (SI) of 2 (indicated by the dotted line) was used as the cutoff value for positive proliferation. Vaccination dates are indicated by light gray arrows and challenge date is indicated by the dark gray arrow. Animals were assessed both during the pre-challenge vaccination phase (panel A) as well as the post-challenge phase (panel B).

FIG. 8. IFN-γ ELISPOT assay. CD8+ cytotoxic T-cell (CTL) responses were assessed by IFN-gamma ELISPOT assay. The frequency of IFN-gamma secreting cells was examined at weeks 6, 12, 20, and 38. A pool of 20 peptides (15-mers) representing conserved regions of the HIV-1 Gag protein were used to stimulate cells. Isolated PBMCs from one HIV-1 seropositive patient served as the positive control. Results in this figure are expressed as the number of IFN-gamma expressing cells per million PBMCs. Representative data from the WOV01 animal group, which was challenged at week 33, is shown.

FIG. 9. Plasma vRNA (viral load) assay. Following SHIV challenge, the levels of plasma SIV RNA were measured by branched DNA (bDNA) assay. The cutoff detection limit for the assay is log 2.1 copies of plasma vRNA per ml (indicated by the dashed line). Representative data from the WOV01 animal group, which was challenged at week 33 (indicated by dark gray arrows), is shown.

FIG. 10. Plasma IgG anti-HIV antibody assay. Serum samples were analyzed for the level of anti-HIV-1 antibody present by enzyme-linked immunosorbent assay (ELISA), using HIV-1 IIIB purified viral lysate as the capture antigen. Vaccination dates are indicated by light gray arrows and challenge dates are indicated by dark gray arrows. Representative data from the WOV01 animal group, which was challenged at week 33, is shown.

FIG. 11. Timeline of prime-boost vaccine trial. Summary of the vaccine trial schedule with the time displayed in weeks post-immunization for groups 1 and 2 (the vaccinated groups) and weeks post-challenge for the control group. Light gray circles represent sample harvest timepoints, dark gray circles represent additional samples taken to accommodate the WOV02 subgroup animals which were challenged at week 39 (as opposed to week 33 for the WOV01 subgroup animals). Light gray arrows indicate time of vaccination, dark gray arrows indicate time of challenge. AT-2: immunization with 500 μl AT-2 inactivated whole-killed virus antigen with 500 μl CpG adjuvant, rAd: immunization with 500 μl rAd antigen with 500 μl CpG adjuvant, SHIV challenge: Viral challenge with combination TCID50=100 of both SHIV 89.6 and SHIV SF162p4.

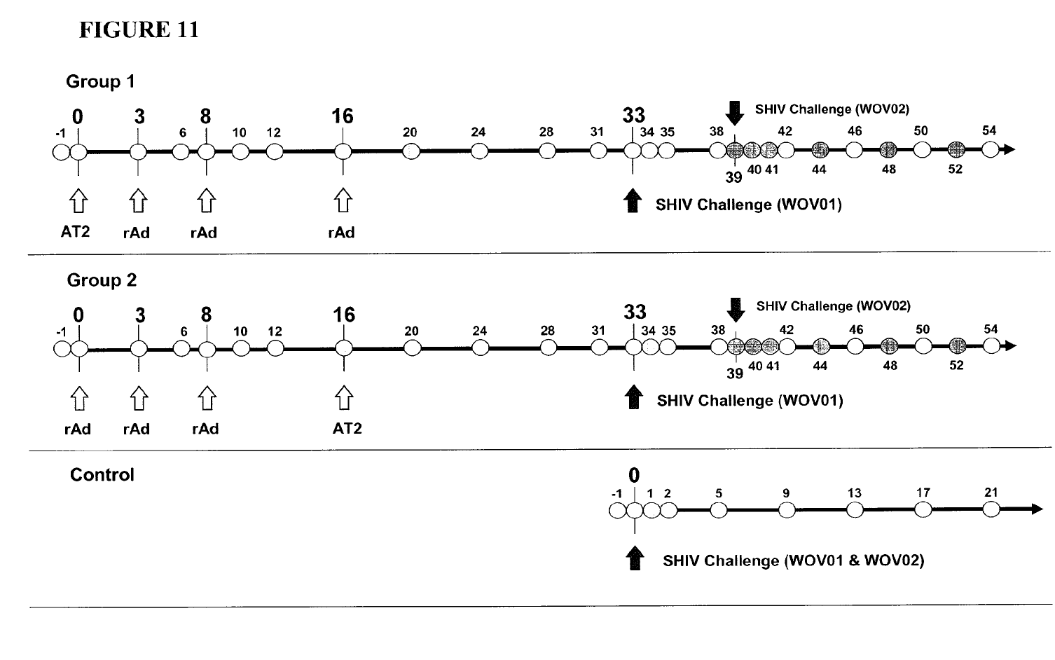

### DETAILED DESCRIPTION

### A. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “administering” includes any method of delivery of a compound of the present invention, including but not limited to, a pharmaceutical composition or therapeutic agent, into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “amino acid” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1 methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (d) and (l) stereoisomers of such amino acids when the structure of the amino acid admits of stereoisomeric forms. The configuration of the amino acids and amino acid residues herein are designated by the appropriate symbols (d), (l) or (dl). Furthermore, when the configuration is not designated the amino acid or residue can have the configuration (d), (l) or (dl). It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (d) or (l) stereoisomers.

The term “antibody” as used herein is intended to include whole antibodies, e.g., of any isotype (IgG, IgA, IgM, IgE, etc), including polyclonal, monoclonal, recombinant and humanized antibodies and fragments thereof which specifically recognize and are able to bind an epitope of a protein. Antibodies can be fragmented using conventional techniques and the fragments screened for utility in the same manner. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFvs may be covalently or non-covalently linked to form antibodies having two or more binding sites.

The terms “comprise” and “comprising” are used in the inclusive, open sense, meaning that additional elements may be included.

The term “conservative substitutions” refers to changes among amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; valine-leucine-isoleucine-methionine; phenylalanine-tyrosine; phenylalanine-tyrosine-tryptophan; lysine-arginine; and histidine-lysine-arginine.

“Equivalent” when used to describe nucleic acids or nucleotide sequences refers to nucleotide sequences encoding functionally equivalent polypeptides. Equivalent nucleotide sequences will include sequences that differ by one or more nucleotide substitution, addition or deletion, such as an allelic variant; and will, therefore, include sequences that differ due to the degeneracy of the genetic code. For example, nucleic acid variants may include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions.

Variant peptides may be covalently prepared by direct chemical synthesis using methods well known in the art. Variants may further include, for example, deletions, insertions or substitutions of residues within the amino acid sequence. Any combination of deletion, insertion, and substitution may also be made to arrive at the final construct, provided that the final construct possesses the desired activity. These variants may be prepared by site-directed mutagenesis, (as exemplified by Adelman et al., DNA 2: 183 (1983)) of the nucleotides in the DNA encoding the peptide molecule thereby producing DNA encoding the variant and thereafter expressing the DNA in recombinant cell culture. The variants typically exhibit the same qualitative biological activity as wild type polypeptides. It is known in the art that one may also synthesize all possible single amino acid substitutions of a known polypeptide (Geysen et al., Proc. Nat. Acad. Sci. (USA) 18:3998-4002 (1984)). While the effects of different substitutions are not always additive, it is reasonable to expect that two favorable or neutral single substitutions at different residue positions in a polypeptide can safely be combined without losing any protein activity. Methods for the preparation of degenerate polypeptides are as described in Rutter, U.S. Pat. No. 5,010,175; Haughter et al., Proc. Nat. Acad. Sci. (USA) 82:5131-5135 (1985); Geysen et al., Proc. Nat. Acad. Sci. (USA) 18:3998-4002 (1984); WO86/06487; and WO86/00991.

In devising a substitution strategy, a person of ordinary skill would determine which residues to vary and which amino acids or classes of amino acids are suitable replacements. One may also take into account studies of sequence variations in families or naturally occurring homologous proteins. Certain amino acid substitutions are more often tolerated than others, and these are often correlated with similarities in size, charge, etc., between the original amino acid and its replacement. Insertions or deletions of amino acids may also be made, as described above. The substitutions are preferably conservative, see, e.g., Schulz et al., Principle of Protein Structure (Springer-Verlag, New York (1978)); and Creighton, Proteins: Structure and Molecular Properties (W. H. Freeman & Co., San Francisco (1983)); both of which are hereby incorporated by reference in their entireties.

The term “essentially noncytolytic” as used herein means that the retrovirus does not significantly damage or kill the cells it infects.

A “functional” fragment of a nucleic acid as used herein is a nucleic acid fragment capable of coding for a signal sequence of a gene linked to the fragment. Thus, a “functional fragment” of a nucleic acid is intended to include nucleic acids capable of coding for a signal sequence in appropriate conditions.

The term “HIV” is known to one skilled in the art to refer to Human Immunodeficiency Virus. There are two types of HIV: HIV-1 and HIV-2. There are many different strains of HIV-1. The strains of HIV-1 can be classified into three groups: the “major” group M, the “outlier” group O and the “new” group N. These three groups may represent three separate introductions of simian immunodeficiency virus into humans. Within the M-group there are at least ten subtypes or clades: e.g., clade A, B, C, D, E, F, G, H, I, J, and K. A “clade” is a group of organisms, such as a species, whose members share homologous features derived from a common ancestor. Any reference to HIV-1 in this application includes all of these strains.

The term “including” is used to mean “including but not limited to”. “Including” and “including but not limited to” are used interchangeably.

The term “non-infectious” means of reduced to non-existent ability to infect.

A “patient” or “subject” or “host” refers to either a human or non-human animal.

The term “pharmaceutical delivery device” refers to any device that may be used to administer a therapeutic agent or agents to a subject. Non-limiting examples of pharmaceutical delivery devices include hypodermic syringes, multichamber syringes, stents, catheters, transcutaneous patches, microneedles, microabraders, and implantable controlled release devices. In one embodiment, the term “pharmaceutical delivery device” refers to a dual-chambered syringe capable of mixing two compounds prior to injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably to refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semi-synthetic, or synthetic origin, which either does not occur in nature or is linked to another polynucleotide in a non-natural arrangement. An “oligonucleotide” refers to a single stranded polynucleotide having less than about 100 nucleotides, less than about, e.g., 75, 50, 25, or 10 nucleotides.

The terms “polypeptide”, “peptide” and “protein” (if single chain) are used interchangeably herein to refer to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

In certain embodiments, polypeptides of the invention may be synthesized chemically, ribosomally in a cell free system, or ribosomally within a cell. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

As known to one skilled in the art, “retroviruses” are diploid positive-strand RNA viruses that replicate through an integrated DNA intermediate (proviral DNA). In particular, upon infection by the RNA virus, the lentiviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells. The retrovirus genome contains at least three genes: gag codes for core and structural proteins of the virus; pol codes for reverse transcriptase, protease and integrase; and env codes for the virus surface proteins. Within the retrovirus family, HIV is classified as a lentivirus, having genetic and morphologic similarities to animal lentiviruses such as those infecting cats (feline immunodeficiency virus), sheep (visna virus), goats (caprine arthritis-encephalitis virus), and non-human primates (simian immunodeficiency virus).

As used herein, “sufficient deletion” means deletion of enough of a nucleic acid sequence to prevent transcription and thereby production of the corresponding protein product.

### B. Methods of Preventing or Treating a Lentiviral Infection

Provided are methods of preventing or treating a lentiviral infection comprising administering (a) an effective amount of a killed recombinant essentially non-infectious avirulent lentivirus of the present invention as a prime injection and (b) an effective amount of a recombinant replication-defective adenovirus vector comprising a nucleic acid encoding a lentiviral protein to an animal in need thereof as a boost immunization modality.

The term “effective amount” as used herein means an amount effective and at dosages and for periods of time necessary to achieve the desired result. The term “animal” as used herein includes all members of the animal kingdom including mammals, preferably humans.

In certain embodiments, (a) is administered to the animal before (b) is administered to the animal.

In certain embodiments, (b) is administered to the patient more than one time over the course of treating or preventing.

In certain embodiments, (a) is administered to the patient in need thereof and (b) is administered to the patient in need thereof at about weeks three, eight and sixteen post-administration of (a).

In certain embodiments, a method of preventing or treating a lentiviral infection comprises administering to a patient in need thereof, (a) an effective amount of a vaccine comprising a recombinant lentivirus having a glycoprotein 120 signal sequence, wherein said glycoprotein 120 signal sequence is selected from the group consisting of the polypeptide sequences listed as SEQ ID NO 3-6, or a functional fragment or variant thereof, wherein said functional fragment or variant thereof contains no more than one (1) positively charged amino acid and (b) an effective amount of a recombinant replication-defective adenovirus vector comprising a nucleic acid encoding a lentiviral protein.

Compositions for use as (a) and (b) in the above methods are further described below.

### C. Compositions for Use as Prime Injection

A variety of killed recombinant essentially non-infectious avirulent lentiviruses wherein the natural signal sequence of the viruses' envelope glycoprotein, preferably gp120, is modified to provide an essentially non-infectious signal sequence, may be used as (a), the prime injection. In certain embodiments, the virus is rendered avirulent by deleting the nef gene.

According to the aforementioned embodiment the modification to provide a non-infectious NSS results in no more than one positively charged amino acid in the NSS sequence. Preferably, the lentivirus is HIV-1.

In certain embodiments, the lentivirus is an essentially noncytolytic recombinant HIV-1 capable of highly efficient replication wherein the NSS of the virus' envelope glycoprotein is replaced with a signal sequence of about 20 to about 40 amino acids in length wherein said signal sequence contains no more than one (1) positively charged amino acids.

The modified gp120 signal sequence can be made by substituting neutral amino acids for positively charged amino acids in the natural signal sequence (MRVKEKKTQHLWRWGWRWGTMLLGMLMICSA; SEQ ID NO: 1); such modifications can be represented as: MX₁VX₂EX₃KTQHLWX₄WGWX₅WGTMLLGMLMICSA (SEQ ID NO: 2) wherein X₁, X₂, X₃, X₄, and X₅ are neutral amino acids. Positively charge residues are shown in bold and underlined.

Exemplary modified signal sequences include:

MRVAEIKTQHLWRWGWRWGTMLLGMLMICSA (YL-1; SEQ ID NO: 3), MIVKEKKTQHLWIWGWIWGTMLLGMLMICSA (YL-2; SEQ ID NO: 4), MRVVEIKTQHLWIWGWIWGTMLLGMLMICSA (YL-3; SEQ ID NO: 5), MIVAEIKTQHLWIWGWIWGTMLLGMLMICSA (YL-4; SEQ ID NO: 6), MKFLVNVALVFMVVYISYIYADPINM (modified melittin signal peptide, the underlined sequence is a result of linker insertion and indicates five amino acids between the signal sequence and the mature gp120 protein; SEQ ID NO: 7), MLLLLLMLFHLGLQASISGRDPINM (modified interleukin 3 signal peptide, the underlined sequence is a result of linker insertion and indicates seven amino acids between the signal sequence and the mature gp120 protein; SEQ ID NO: 8), or a functional fragment or variant thereof.

Other compositions and methods for producing such compositions are described in U.S. Pat. No. 7,067,134, which is incorporated herein by reference in its entirety.

The recombinant lentiviruses of the present invention can be prepared using techniques known in the art. In one embodiment, the lentivirus may be introduced in a host cell under conditions suitable for the replication and expression of the lentivirus in the host.

Accordingly, the present invention also provides a cell transfected with a recombinant lentivirus wherein the natural signal sequence of the virus' envelope glycoprotein gp120 is modified to provide an essentially non-cytotoxic virus or is replaced with an essentially non-infectious signal sequence. The cell is preferably a T-lymphocyte, more preferably a T-cell that is not derived from a transformed cell line.

The present invention further features methods comprising the administration of an effective amount of an avirulent and an essentially non-infectious lentivirus as described above. Dosage levels of between about 0.01 and about 2.5 mg/kg body weight, preferably between about 0.05 and about 0.5 mg/kg body weight, and most preferably between about 0.10 and about 0.23 mg/kg body weight are useful as a prime injection in the methods described herein. The amount of active ingredient that may be combined with the carrier materials to produce a single dosage form will vary depending upon the host treated and the particular mode of administration. The dose of the vaccine may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances.

The compositions of the invention are suitable for administration to subjects in a biologically compatible form in vivo. The expression “biologically compatible form suitable for administration in vivo” as used herein means a form of the substance to be administered in which any toxic effects are outweighed by the therapeutic effects. The substances may be administered to any animal, preferably humans.

The vaccines of the present invention may additionally contain suitable diluents, adjuvants and/or carriers. Preferably, the vaccines contain an adjuvant which can enhance the immunogenicity of the vaccine in vivo. The adjuvant may be selected from many known adjuvants in the art including the lipid-A portion of gram negative bacteria endotoxin, trehalose dimycolate of mycobacteria, the phospholipid lysolecithin, dimethyldictadecyl ammonium bromide (DDA), certain linear polyoxypropylene-polyoxyethylene (POP-POE) block polymers, aluminum hydroxide, liposomes and CpG (cytosine-phosphate-guanidine) polymers. The vaccines may also include cytokines that are known to enhance the immune response including GM-CSF, IL-2, IL-12, TNF and IFNγ.

The vaccines of the instant invention may be formulated and introduced as a vaccine through oral, intradermal, intramuscular, intraperitoneal, intravenous, subcutaneous, intranasal, and intravaginal, or any other standard route of immunization.

In formulations of the subject vaccines, wetting agents, emulsifiers and lubricants, such as sodium lauryl sulfate and magnesium stearate, as well as coloring agents, releasing agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any method well known in the art of pharmacy. The amount of composition that may be combined with a carrier material to produce a single dose may vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations, which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants, which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

In addition, vaccines may be administered parenterally as injections (intravenous, intramuscular or subcutaneous). The vaccine compositions of the present invention may optionally contain one or more adjuvants. Any suitable adjuvant can be used, such as CpG polymers, aluminum hydroxide, aluminum phosphate, plant and animal oils, and the like, with the amount of adjuvant depending on the nature of the particular adjuvant employed. In addition, the anti-infective vaccine compositions may also contain at least one stabilizer, such as carbohydrates such as sorbitol, mannitol, starch, sucrose, dextrin, and glucose, as well as proteins such as albumin or casein, and buffers such as alkali metal phosphates and the like.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers, which may be employed in the pharmaceutical compositions of the invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Further, non-infectious recombinant lentivirus of the present invention may be encapsulated in liposomes and administered via injection. Commercially available liposome delivery systems are available from Novavax, Inc. of Rockville, Md., commercially available under the name Novasomes™. These liposomes are specifically formulated for immunogen or antibody delivery. In an embodiment of the invention, Novasomes™ containing Isd peptides or antibody molecules bound to the surface of these non-phospholipid positively charged liposomes may be used.

### D. Compositions for Use as Boost Immunization Modalities

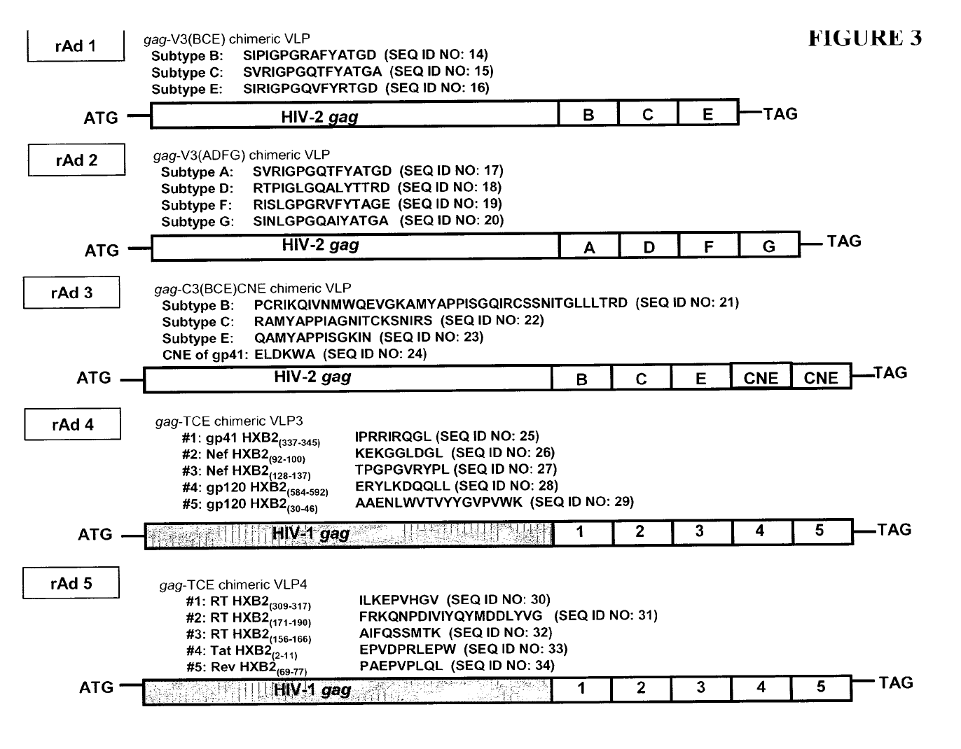

A variety of replication-defective recombinant adenoviral vectors based on the adenovirus type 5 (Ad5) genome may be used as (b), the boost immunization modality, in the methods described above. That is, the backbone vector consisting of the Ad5 genome contains a deletion of the adenovirus E1A gene, which is required for viral replication (Graham, F., et al. (1977) General Virology 36:59-72). It is in this deleted gene region that our target HIV-1 genes (described below, and in FIG. 3) have been inserted. Target HIV-1 genes may be selected from the Los Alamos National Laboratory HIV Databases at http://www.hiv.lanl.gov/content/index.

In certain embodiments, a HIV gene is inserted in said E1A region. The HIV gene may be, for example, HIV-1 gag or HIV-2 gag.

In other embodiments, a HIV gene and at least one neutralizing or T-cell epitope is inserted in the E1A region. The at least one neutralizing or T-cell epitope may be selected, for example, from the group consisting of any of SEQ ID NOs: 14 through 34.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100 200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the E3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Methods for culturing 293 cells and propagating adenovirus may include growing natural cell aggregates by inoculating individual cells into 1 liter siliconized spinner flasks (Techne, Cambridge, UK) containing 100 200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 hours.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression and vaccine development. Recently, animal studies suggested that recombinant adenovirus could be used for gene therapy (Stratford-Perricaudet and Perricaudet, (1991) In: Human Gene Transfer, Eds, O. Cohen-Haguenauer and M. Boiron, Editions John Libbey Eurotext, France, pp. 51 61; Stratford-Perricaudet et al. (1990) Hum. Gene Ther., 1:241 256; and Rich et al. (1993) Hum. Gene Ther., 4:461 476). Studies in administering recombinant adenovirus to different tissues include trachea instillation, muscle injection, peripheral intravenous injections and stereotactic inoculation into the brain.

The present invention further features methods comprising the administration of an effective amount of the replication-defective recombinant adenoviral vectors based on the adenovirus type 5 (Ad5) genome as described above. Dosage levels of between about 1×10⁸ and 1×10¹⁰ pfu/kg body weight, preferably between about 5×10⁸ and 5×10⁹ and most preferably between about 8.46×10⁸ and 2.21×10⁹ pfu/kg body weight are useful as a boost immunization modality in the methods described herein. The amount of active ingredient that may be combined with the carrier materials to produce a single dosage form will vary depending upon the host treated and the particular mode of administration. The dose of the vaccine may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances.

### E. Kits

The present invention provides kits, for example for preventing or treating a lentiviral infection. For example, a kit may comprise one or more pharmaceutical compositions as described above and optionally instructions for their use. In still other embodiments, the invention provides kits comprising one or more pharmaceutical compositions and one or more devices for accomplishing administration of such compositions.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

### EXAMPLES

The present invention is further illustrated by the following examples which should not be construed as limiting in any way. The contents of all cited references including literature references, issued patents, published or non published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### Example 1

### Genetically Modified, Attenuated HIV-1 Capable of High Titre Replication

In the case of HIV, a number of stumbling blocks have prevented development of whole-killed or inactivated viruses as a vaccine including the inability of scientists to safely produce large quantities of the virus for inactivation, given that attenuation of virulence commonly results in a decrease in viral replication. Our approach, which overcomes this problem, is the construction of a non-cytotoxic, avirulent HIV-1 capable of high-titre replication, and is based upon modification of two of the viral proteins, Nef and Env.

The nef gene of HIV-1 encodes a 210-250 amino acid protein, 25-27 kDa in size. A number of functions have been attributed to Nef, including downregulation of cell surface proteins such as CD4 and MHC class I molecules. Presumably, reduction of available CD4 molecules on the cell surface acts to prevent superinfection of cells, while removal of MHC I complexes represents one of the viral immune evasion strategies. In addition, Nef has been shown to play an important role in viral infectivity, and also, to modulate host cell signal transduction pathways, via interaction with protein kinases. The most important function ascribed to Nef however, as it relates to the construction of this genetically-modified virus, is its role in HIV-1 pathogenicity. Several lines of evidence indicate that the Nef protein of HIV and other primate lentiviruses is critical for viral pathogenesis. In the related virus, SIV, it has been shown that not only are nef-deleted strains non-pathogenic, but that infection with these viruses confers protection against subsequent, wild type viral challenge. Additionally, experiments in the severe combined immunodeficient mouse model (SCID-hu) of human HIV-1 infection, have shown that Nef is required for effective in vivo pathogenicity. Even more compelling than these reports however, has been the identification of long-term non-progressive AIDS patients who are infected with nef-defective strains of HIV-. These individuals, while infected with HIV-1, fail to progress to full-blown AIDS (or do so over a greatly extended time course), and genetic analysis indicates that the only significant alteration in the infecting virus, is a disruption of the nef gene. Taken together, this evidence suggests that HIV-1 strains containing a targeted deletion of the nef gene would exhibit an avirulent, or strongly attenuated phenotype.

The env gene is the other gene of interest with regards to construction of our genetically modified HIV-1, or more specifically, the signal sequence of the Env glycoprotein. The Env protein is originally synthesized as a heavily glycosylated, 160 kDa precursor, consisting of approximately 850 amino acids. This polyprotein is subsequently cleaved by host endopeptidase into the surface glycoprotein, gp120, and transmembrane protein, gp41, which are responsible for cell attachment and viral entry, respectively. One unusual feature of the Env protein is its unusual signal sequence. All signal sequences are essentially built along the same general lines. That is, they possess a positively charged N-terminal region, a central hydrophobic region, and a polar C-terminal region that defines the cleavage site. The HIV-1 Env protein natural signal sequence (EnvNSS) however, contains an unusually long hydrophobic domain, and a highly positively charged N-terminus. Previously, we have shown that replacement of the EnvNSS with that of honeybee melittin (EnvMSS) resulted in increased Env protein expression and secretion, rapid dissociation from the molecular chaperone calnexin, and efficient signal sequence cleavage. In addition, we observed accelerated kinetics of glycoprotein folding and intracellular transport when the EnvNSS was replaced. More importantly however, was the discovery that the presence of the EnvNSS was responsible for both cellular apoptosis and necrosis. It was found that recombinant gp120, expressed from its natural signal sequence killed cells rapidly, while replacement with the melittin signal sequence abrogated this effect. Overall, this data suggests that replacement of the HIV-1 EnvNSS with another more efficient and non-cytotoxic signal sequence (such as that of honeybee melittin) would result in the production of HIV-1s which would possess a non-cytotoxic phenotype, and exhibit an enhanced replicative capacity.

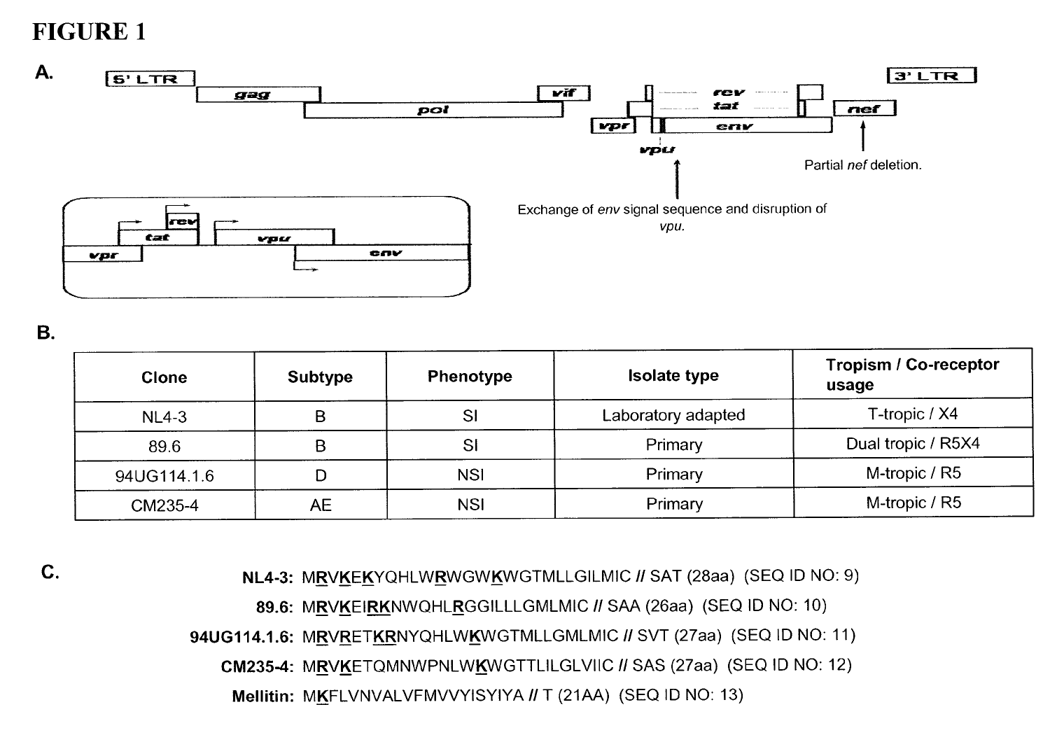

Taken together then, combination of a targeted deletion of the nef gene, combined with replacement of the EnvNSS with that of honeybee melittin, should result in a virus with reduced pathogenicity, while remaining capable of rapid, high-titre replication in infected cells. To this end, our laboratory first constructed a genetically modified HIV-1 which combines these mutations in the highly studied and well characterized provirus, pNL4-3. HIV-1_{NL4-3} is a laboratory-adapted subtype B virus which exhibits T-cell tropism and strong syncitium-inducing ability (FIG. 1B). The pNL4-3 provirus then, is an infectious molecular clone of the HIV-1_{NL4-3} strain, available through the NIH AIDS Research and Reference Reagent program suitable for genetic modification.

Using this provirus we first constructed a targeted deletion of the nef gene to reduce viral pathogenicity (FIG. 1A). The deletion was generated by restriction enzyme digestion resulting in the removal of 206 nucleotides downstream of the Nef initiation codon, but upstream of the HIV Long Terminal Repeat (LTR) which is critical for viral replication. This deletion not only removes several internal regions important for Nef function, but also induces a series of premature stop codons which severely truncates the protein. The end result is a coding region of only 18 amino acids, which we believe results in the production of a non-functional protein which is rapidly degraded by the host cell as we cannot detect its presence by Western blot analysis.

The second modification of the provirus was replacement of the EnvNSS with honeybee melittin to reduce cytotoxicity and increase the efficiency of viral replication. As stated above, HIV-1 contains an unusually long, highly positively charged signal sequence. The pNL4-3 EnvNSS is 28 amino acids in length, and contains five positively-charged amino acids (FIG. 1C). Using PCR and molecular cloning techniques, this signal sequence was replaced with the highly-efficient honeybee melittin signal sequence, which is 21 amino acids in length and contains only a single positively-charged amino acid. It is important to note that the N-terminus of the HIV-1 env gene, where the EnvNSS is located, overlaps with the C-terminus of the HIV-1 vpu gene in the viral genome (FIG. 1A). Fortunately, the HIV-1 vpu gene, while playing a role in viral infectivity, has been shown to be dispensable for viral replication, and as you will see, does not limit propagation of the virus.

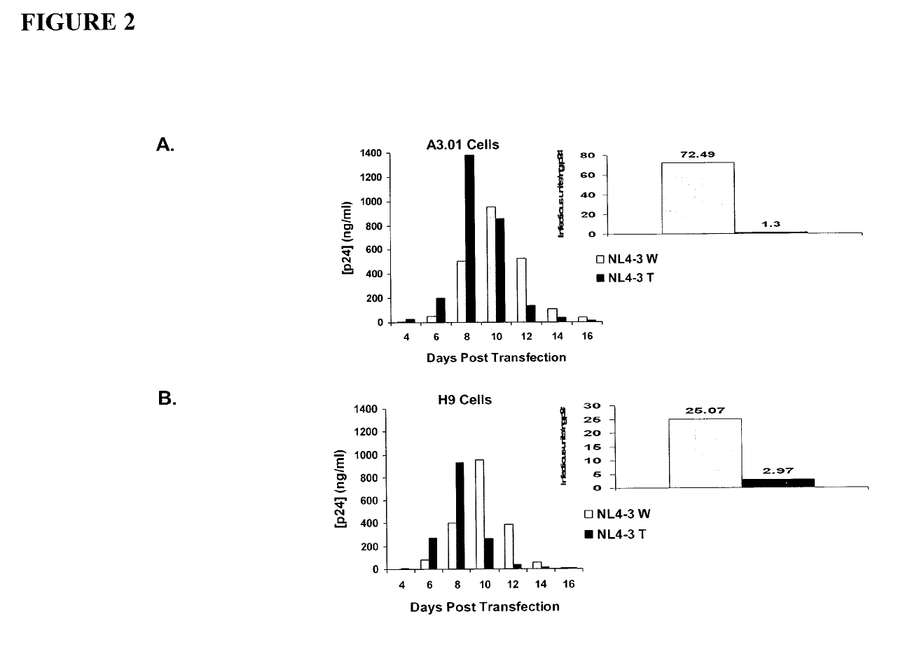

Once constructed, both the wild-type (NL4-3 W) and genetically-modified (NL4-3 T) viruses were recovered by transfection of the proviral DNA into the susceptible T-cell lines A3.01 and H9 which support HIV-1 replication. Once transfected into susceptible cells, the proviral DNA clones immediately begin to express their encoded HIV-1 gene products resulting in the production of progeny virus particles which can be harvested and used in subsequent experiments. Following transfection of the infectious molecular clones, cells were cultured and split every 2 days with samples of the culture supernatant collected and analyzed by p24 ELISA starting at day 4 post-transfection in order to monitor viral replication. This experiment measures the amount of p24 antigen released into the culture supernatant by infected cells. This is a widely accepted assay to indirectly measure the level of HIV-1 replication. In both A3.01 and H9 cells, the genetically modified NL4-3 T virus replicated to the same or higher titre than wild-type HIV-1, and did so with noticeably accelerated kinetics with the peak of viral replication in NL4-3 T being reached at 96 hours post transfection, 48 hours earlier than in the wild-type NL4-3 W (FIG. 2).

To further characterize these viruses, samples were also analyzed by multi-nuclear activator of a galactosidase indicator (MAGI) assay, which assesses viral infectivity. In this system a HeLa-based cell line which expresses CD4 (the viral receptor) on its cell surface is infected with various dilutions of the virus. Once inside the cell, replicating virus will begin producing viral proteins including the viral transactivator protein, Tat. Tat subsequently activates expression of a B-galactosidase gene driven by the viral LTR promoter which has been introduced into the cellular genome. The B-galactosidase enzyme produced within infected cells then goes on to cleave the substrate X-gal which is supplied to the cells, resulting in development of a blue color. Thus, blue cells are counted as being ‘infected’, and by calculating the number of blue cells relative to the dilution of the virus, the overall number of infectious viral particles present can be determined. Results of these experiments indicated that despite its ability to rapidly replicate to high-titre, the NL4-3 T virus was rendered 10- to 50-fold less infectious than the wild-type (FIG. 2 inset). These results confirm that it is indeed possible to generate an attenuated HIV-1 strain capable of high-titre replication through genetic modification.

To further support these results and confirm that this phenomena is general to HIV-1 biology and not simply to the strain we had selected (NL4-3), similar constructs were generated in several other HIV-1 strains including 89.6, CM235-4 and 94UG114.1.6 (FIGS. 1B and C). These represent not only a variety of HIV-1 subtypes, but also variations in syncitium induction, isolate source, and cellular tropism. In short, all viruses that have been recovered behave similarly to NL4-3, with the genetically-modified viruses which contain the combination nef deletion and EnvNSS replacement replicating more efficiently than their wild-type counterparts, despite exhibiting strongly reduced infectivity. HIV_{89.6} for example, is a subtype B, syncitium inducing, dual-tropic isolate of HIV-1. The mutant 89.6 T virus, when transfected into susceptible A3.01 cells, replicates much more efficiently than wild-type virus, peaking at over 1000 ng/ml p24 (comparable to the laboratory-adapted NL4-3 strain) whereas wild-type virus, despite being 10-fold more infectious than the modified virus reached a peak of less than 600 ng/ml p24. These results confirm our hypothesis that the combination of HIV-1 nef gene deletion and Env signal sequence replacement results in an efficiently replicating HIV-1 with strongly attenuated infectivity. The ability to construct these types of modified viruses from multiple HIV-1 subtypes is important, as it may be necessary to combine multiple HIV-1 subtypes into a single vaccine formulation in order to provide protection against the growing number of HIV strains present in the world today.

### Example 2

### Replication-Defective Recombinant Adenovirus and VLPs

Adenovirus vectors have several qualities that make them attractive as vaccine vectors. They replicate rapidly to high titre in permissive cell lines, and are capable of producing large quantities of the protein of interest. They are capable of infecting both dividing and non-dividing cells and are episomal in nature and thus do not integrate into the host genome (this minimizes the risk of transformation and potential oncogenic effects). They are capable of targeting foreign genes to many sites including the mucosa, gastrointestinal tract and to organs or tissues parenterally, thereby inducing both mucosal and systemic immunity. Additionally, military and civilian vaccination programs which utilized enteric-coated capsules of the more virulent adenovirus types 4 and 7 in unattenuated, fully replication-competent forms have previously established the safety of Ad vaccine vectors. It is important to note however, that current generation adenovirus vectors, including those used in this study have been engineered to be replication-defective viruses capable of single-round replication only.

Our system utilizes replication-defective recombinant adenoviral vectors based on the adenovirus type 5 (Ad5) genome. That is, the backbone vector consisting of the Ad5 genome contains a deletion of the adenovirus E1A gene, which is required for viral replication. It is in this deleted gene region that our target HIV-1 genes (described below, and in FIG. 3) have been inserted. These recombinant viruses can be propagated to high titre in vitro in a permissive cell line (e.g. 293 cells) which provides the E1A protein in trans, but outside of the producer cell-line are incapable of replication beyond a single cycle (meaning that, in a vaccinated host in vivo, the adenovirus vector is capable of entering the cell and producing the desired protein, but does not produce progeny adenovirus particles). This provides an additional measure of safety and control to the rAd system.

For use in this study, our laboratory has generated a total of 5 replication-defective rAd vectors. Each vector consists of the E1A-deleted Ad5 backbone into which has been inserted the gag gene of either HIV-1 or HIV-2, and a series of HIV-1 specific neutralizing or T-cell epitopes selected from different regions of the virus (FIG. 3).

The gag gene of HIV-1 typically produces a 55 kDa polyprotein, which is subsequently cleaved into the viral capsid (p24), matrix (p17) and p6/9 structural proteins by the viral protease, which is also encoded in this region. However, our laboratory has discovered that deletion of the region of gag which encodes the viral protease, and its subsequent expression from rAd vectors allows for the formation of virus-like particles (VLPs) in rAd infected cells. Thus, when infected with replication-defective rAd particles containing the truncated HIV gag gene (rAd-Gag), not only is the Gag protein produced, but it is capable of then self-assembling into virion-like structures which are subsequently secreted from the infected cell, despite the lack of any viral replication by the adenovirus vector. Further, selected HIV epitopes can be incorporated and fused into this deleted region (rAd-Gag-polyepitope), which when expressed in host cells generates VLPs which possess not only the HIV Gag epitopes but the selected neutralizing or T-cell epitopes as well. This cycle; infection—protein production—VLP formation—release, represents an important aspect of the immunogenicity of this type of vaccination.

Typically, when an antigen is expressed internally within a host cell (e.g. from a rAd vector), it is processed by the cell and presented to the immune system via the major histocompatability class I (MHC I) system which is involved in eliciting a type 1 or cell-mediated cytotoxic T-lymphocyte (CTL) response. This type of response has been suggested as being vital to the control of the initial stages of HIV infection as it results in the elimination of infected cells. Conversely, when a host cell receives an antigen exogenously (e.g. in the form of a VLP), it is processed by the cell and presented to the immune system via the MHC II system which is involved in eliciting a type 2 or antibody-mediated humoral response. This type of response is important in the generation of neutralizing antibodies to prevent the initial HIV infection of cells as well as the antibody-mediated targeting of viral particles for elimination. Thus, the rAd system has the ability to generate not only cellular, but humoral immunity, both of which are expected to play important roles in the generation of a protective immune response against HIV-1 infection.

The panel of replication-defective rAd vectors produced for these experiments can be divided into 2 categories, those which contain HIV-1 neutralizing epitopes, and those which contain HIV-1 CTL epitopes. The rAd vectors 1, 2 and 3 contain the HIV-2 gag gene fused with neutralizing epitopes (designed to enhance the humoral response) from both the gp120 variable region 3 and constant region 3 from a number of HIV-1 subtypes. Additionally, rAd vector 3 contains the conserved neutralizing epitope (CNE) of gp41, the viral fusion protein. The rAd vectors 4 and 5 contain the HIV-1 gag gene fused with T-cell epitopes (designed to enhance the cellular immune response) of the subtype B HIV_{HXB2} virus, selected from several viral proteins including Tat, Rev, Nef, the viral reverse transcriptase (RT) and gp120 glycoprotein. These replication-defective rAd vectors, originally constructed as dsDNA plasmids, were transfected into helper 293 cells which supply the E1A gene required for adenoviral replication in order to recover the infectious rAd particles. The recovered viruses were then screened by DNA sequencing and protein expression analysis to confirm their expression of the HIV-1 Gag-polyepitope fusion protein. By incorporating these selected immunodominant epitopes into the rAd vectors which cover not only a broad range of HIV-1 subtypes, but viral protein targets as well, we optimize the ability of the vaccine to elicit an immune response against not just one, but many strains of HIV. This is again, an important consideration for a vaccine that must protect against a virus which can exist as several species even within a single infected individual.

### Example 3

### Vaccination Strategy

The overall vaccination strategy taken here is a two-fold prime-boost approach. In a prime-boost system, the host is exposed to first one type of antigen/vector, followed by another (e.g. in our system, inactivated whole-killed virus and replication-defective rAd). This type of approach challenges the immune system with not only different viral epitopes, but utilizes different routes and presentation pathways to do so. This has been shown to result in a more robust immunity, amplifying the response of both the humoral and cellular arms of the immune system. As well, depending upon the route of administration, mucosal immunity can also be developed. Combined with an effective adjuvant, the prime-boost vaccination approach has been shown to be capable of stimulating a much stronger and broader immune response than via repeated vaccination with either of its component antigen/vectors alone.

### Example 4

### Vaccine Formulation

In the experiments described here, the two components to our prime-boost strategy include 1) inactivated whole-killed virus antigen, and 2) replication-defective rAd vectors.

In order to prepare our inactivated whole-killed virus antigen, our genetically modified HIV-1 NL4-3 T virus was used to infect A3.01 cells (a human T-cell line). The virus was grown to high titre, expanding cultures and replacing media every 2 days. Beginning at 8 days post infection, virus-containing supernatants were harvested, and fresh media and uninfected A3.01 cells were added to infected cell cultures to continue and maintain virus production. This continued every 48 hours until 16 days post infection when all culture supernatants were pooled. The virus-containing supernatant was clarified of cellular debris once via centrifugation at 700×g for 10 minutes, and then again by passage through a 0.45 μm filter. This clarified supernatant was then subjected to ultracentrifugation to pellet and concentrate the virus. The now virus-free supernatant was removed, and the pellet resuspended in a small volume of PBS to which the chemical agent aldrithiol-2 (AT-2) was added at a final concentration of 1000 μM. AT-2 treatment has been shown to be a robust method of retroviral inactivation. It acts by modifying the zinc-finger domains of the viral nucleocapsid protein resulting in ejection of the coordinated zinc and loss of infectivity (virus is able to bind and enter cells, but unable to begin the reverse transcription process. Unlike other inactivation methods such as heat-exposure or formalin treatment, AT-2 inactivation carries with it the added benefit of having no negative effect on the structure and conformation of the viral glycoproteins, which remain completely intact. For this reason and the continual effort and evaluation in establishing AT-2 as the inactivation method of choice for retroviruses, the compound was selected for this purpose in our experiments. The AT-2 treated virus stock was incubated for 1 hour at 37° C. to allow for complete virus inactivation. The virus was then layered upon a 20% sucrose cushion and again ultracentrifuged to further concentrate the virus and separate it from residual proteins and chemical contaminants (such as AT-2). Virus was resuspended at a final concentration of 1 mg/ml in 500 μl aliquots and stored at a temperature of −80° C. until ready for use in the vaccination protocol (thus each aliquot contained 500 μg total viral protein in 500 ul PBS). It is important to note that several aliquots of the inactivated virus stock were taken and tested by MAGI assay to determine if any residual infectivity remained. In each sample tested, virus infectivity was completely eliminated with no sign of contaminating infectious virus.

In order to prepare our replication-defective rAd virus stocks, each of the 5 vectors was used to infect permissive 293 cell cultures and was grown to high titre. Infected cells were harvested, lysed, and the virus particles purified by banding via ultracentrifugation through a CsCl gradient. Viral bands were isolated, and residual CsCl removed via extensive dialysis against PBS²⁺ with 10% Glycerol. This stock virus was then titrated, and resuspended at a final concentration of 1×10¹⁰ infectious particles/ml. From these, 500 μl aliquots were made, each containing 100 μl from each of the 5 viral stocks (thus each aliquot contained 1×10⁹ infectious particles of each of the 5 rAds for a total of 5×10⁹ infectious particles in 500 μl PBS²⁺ with 10% glycerol). These aliquots were then stored at −80° C. until ready for use in the vaccination protocol. To ensure that the replication-defective rAd particles remained infectious after freezing at −80° C., samples from both the individual virus stocks, and aliquoted mixed-virus suspensions were tested for infectivity by plaque assay on E1A expressing 293 cells. No loss of infectivity was observed in the any of the frozen rAd stocks.

### Example 5

### Adjuvant Selection

An important part of vaccine formulation is the selection of an appropriate adjuvant. An adjuvant, although not necessarily eliciting an immune response itself, acts to enhance the immune response to a co-administered antigen. Adjuvants can have many effects such as raising antibody titres, improving CTL responses or enhancing mucosal immunity. Indeed, depending upon the adjuvant selected the immune response generated to a particular antigen, it may be swung in different directions. For example, the primary adjuvant currently licensed for use in humans is Alum, which pushes the immune system towards a type 2 antibody-mediated response. However, for our purposes alum provides a relatively weak adjuvant effect and an antibody response alone is unlikely to be protective against retrovirus infection. A relatively new alternative to traditional vaccine adjuvants is the development of so-called CpG motifs or immunostimulatory oligodeoxynucleotides (ODNs). CpG motifs are short stretches of immunostimulatory bacterial DNA of defined sequence. These act by stimulating the host's innate immune system to augment the immune response against the target antigen. Unlike alum, CpG DNA is capable of inducing a much stronger immunological reaction directed not only at stimulating the development of an antibody-mediated response, but a strong CTL response as well (which is believed to be particularly important in controlling HIV infection). Panels of various sequences of CpG motifs have been tested and optimized for their efficacy in non-human primate hosts, and are commercially available. For its ability to elicit both humoral and cellular immune responses in non-human primates including rhesus macaques, the CpG ODN of sequence 5′-TCGTCGTTTTGTCGTTTTGTCGTT-3′ was selected for use as adjuvant in these experiments. Note that the ODNs used here were synthesized on a phosphorothioate backbone to prevent them from host nuclease digestion, thus prolonging their in vivo half-life.

### Example 6

### Vaccination Schedule and Experimental Outline

The test subjects for this vaccine study were 18 male rhesus macaques (Macaca mulatto) which were housed at the California Regional Primate Research Center at the University of California at Davis.

Two types of antigen were used in the prime-boost approach vaccination strategy, both of which were combined with CpG ODN adjuvant prior to their administration into the host animals:

AT-2 inactivated whole-killed virus antigen: Genetically modified HIV-1 NL4-3 T virus which has been produced, purified and undergone AT-2 inactivation. For immunization, specified animals will receive 500 μg of antigen suspended in 500 μl PBS (formulated with 500 μl of adjuvant).

Replication-defective recombinant Adenovirus antigen (rAd antigen): High-titre stocks of five rAd vectors expressing the HIV-1 gag gene in association with a number of selected neutralizing and T-cell epitopes have been prepared and purified. For immunization, specified animals will receive 1×10⁹ infectious units of each recombinant virus (1×10⁹ infectious units×5 recombinant viruses=5×10⁹ infectious units) in a total volume of 500 μl (formulated with 500 μl of adjuvant).

CpG oligodeoxynucleotide (ODN) adjuvant: Purified phosphorothioate oligodeoxynucleotides of the sequence 5′-TCGTCGTTTTGTCGTTTTGTCGTT-3′ obtained from Coley pharmaceuticals. 500 μg of this ODN will be suspended in a total volume of 500 μl PBS for formulation with each antigen described above.

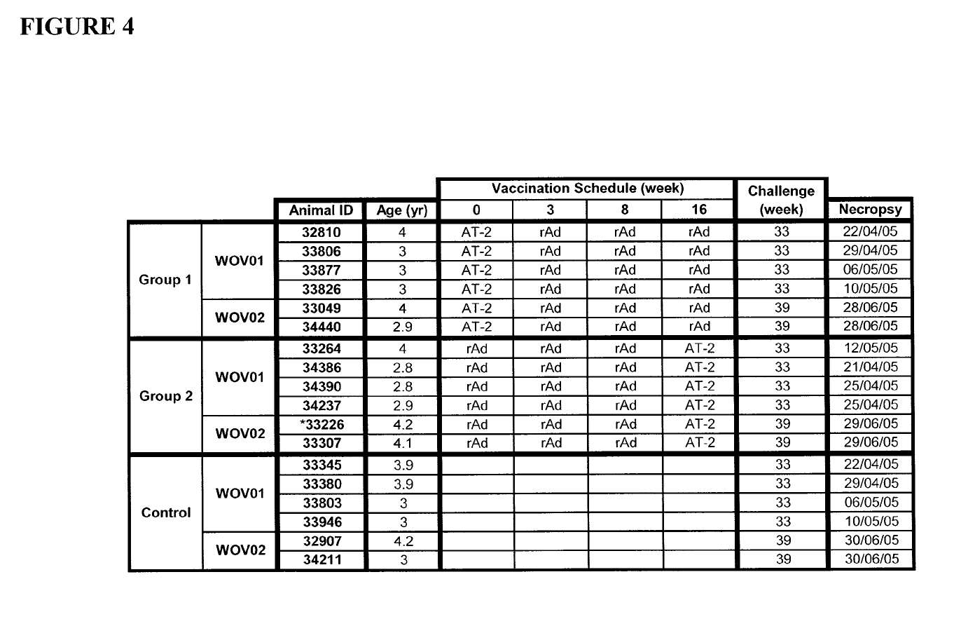

As outlined in FIG. 4, animals were subsequently divided into 3 groups (designated Group 1, 2 and Control), with each group containing a total of 6 macaques. The immunization schedule for each group of animals is listed below including time of inoculation, type and quantity of antigen/adjuvant. All immunizations were administered intramuscularly.

Group 1
Week 0-500 μl inactivated whole-killed virus antigen with 500 μl CpG adjuvant
Week 3-500 μl rAd antigen with 500 μl CpG adjuvant
Week 8-500 μl rAd antigen with 500 μl CpG adjuvant
Week 16-500 μl rAd antigen with 500 μl CpG adjuvant

Group 2
Week 0-500 μl rAd antigen with 500 μl CpG adjuvant
Week 3-500 μl rAd antigen with 500 μl CpG adjuvant
Week 8-500 μl rAd antigen with 500 μl CpG adjuvant
Week 16-500 μl inactivated whole-killed virus antigen with 500 μl CpG adjuvant

Control
Control animals, received no prior antigenic exposure to either the HIV antigens or challenge virus.

Following vaccination, animals were further subdivided based on date of challenge (FIG. 4). An initial group of 12 animals (4 each from group 1, 2 and control—designated WOV01), were challenged intravenously at 33 weeks post primary immunization with hybrid simian-human immunodeficiency virus (SHIV). The SHIV challenge consisted of a combined infection of SHIV_{89.6} and SHIV_{SF162p4} administered at a tissue culture infectious dose 50 (TCID₅₀) of 100 for each virus. The remaining 6 animals (2 each from group 1, 2 and control—designated WOV02), were challenged at 39 weeks post primary immunization with the same combination SHIV inoculum.

This separation of groups was necessary to accommodate animal 33226. At its medical examination prior to the week 33 challenge, animal 33226 showed some clinical symptoms of rhesus arthritis including an elevated CBC count. Although unrelated to the vaccination protocol, the condition could affect immunological results and challenge outcome. After consultation with the attending veterinarian it was decided to delay challenge of this animal for 6 weeks to monitor its condition. For statistical reasons, 2 animals from each group (including 33226 from group 2) were held back and challenged at week 39, while the remaining animals were challenged as scheduled at week 33.

Blood samples were taken from each animal both pre- and post-immunization, and monthly thereafter until challenge to assess the immune response to the vaccination protocol. Further, to assess the immune response to viral challenge as well as monitor viral load and potential disease progression, samples were taken at weeks 1, 2 and 5 post-challenge and monthly thereafter. Animals were euthanized and necropsies performed approximately 6 months post-challenge, and additional samples collected including blood, spleen and axillary lymph nodes.

### Example 7

### Animal Health and Vaccine Tolerance

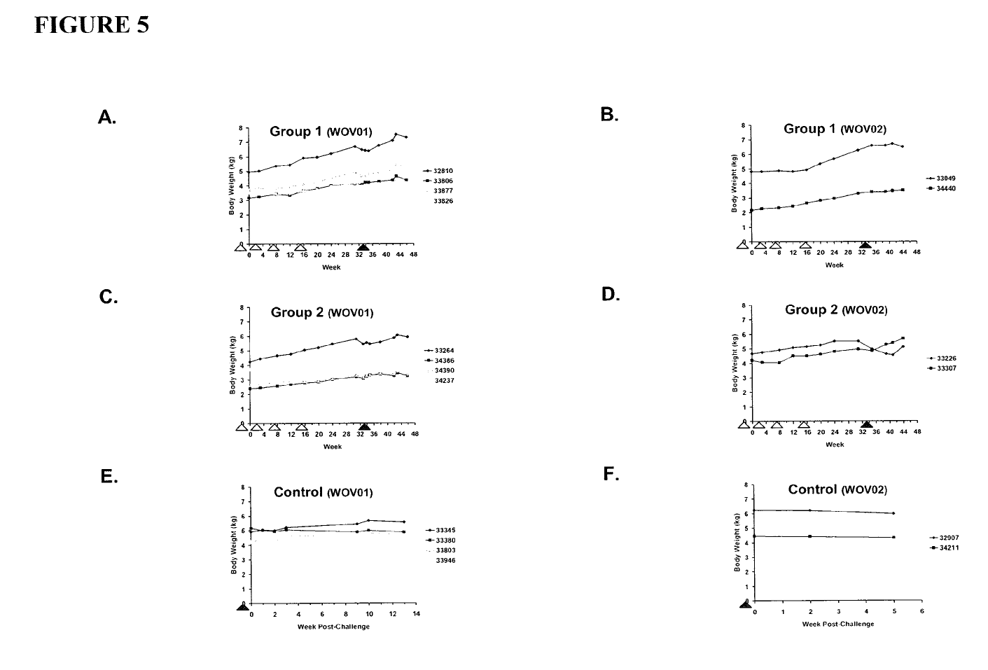

Over the course of the study all animals were regularly monitored to assess their health and well being. This included a general physical examination as well as periodic measurement of body weight. All animals tolerated vaccination and challenge well, with no measurable untoward side-effects. As shown in FIG. 5 (A-D), all animals from group 1 and 2 showed a steady increase in body weight throughout the vaccinations at weeks 0, 3, 8, and 16, and on to challenge. Immediately post-challenge some animals in the two vaccinated groups showed a slight drop in body weight (<0.5 kg), however they recovered quickly and continued to remain healthy with a steady increase in body weight until necropsy. Similarly, some of the control animals for both the WOV01 (FIG. 5E) and WOV02 (FIG. 5F) subgroups showed a slight fluctuation in body weight immediately post-challenge, but recovered and maintained a steady body weight until necropsy.

Prior to the scheduled challenge at week 33, one animal (33226) was identified as exhibiting symptoms of a condition commonly referred to as rhesus arthritis. This is not an uncommon condition among rhesus macaques and should not be considered to have occurred or been induced by the vaccination protocol. Because of this condition, the animal was monitored until week 39, whereupon consultation with the attending veterinarian it was deemed fit to continue with the study and was challenged along with the 5 other remaining animals of subgroup WOV02.

All animals were euthanized approximately 6 months post-challenge, and necropsies were performed. The animals showed no gross lesions or enlargement of lymph nodes. There were no signs of weight loss or abnormalities in complete blood count (CBC).

Overall, the results suggest that the protocols laid out for both group 1 and group 2 were safe, as all vaccinations were well-tolerated, and no negative side-effects were observed. Animals remained healthy over the course of the experiment with no significant issues or signs of weight-loss.

### Example 8

### Clinical Signs of Disease Progression

Although both of the challenge viruses used (SHIV_{89.6} and SHIV_{SF162p4}) were non-pathogenic strains, the levels of CD4+ and CD8+ cells in the blood were measured and the CD4:CD8 ratio monitored to determine whether any animals showed signs of clinical disease progression. Healthy animals normally maintain a CD4:CD8 ratio ≧1, whereas animals progressing to simian AIDS as a result of SIV or SHIV infection can show a marked decline in CD4+ cells and thus a decreased CD4:CD8 ratio.

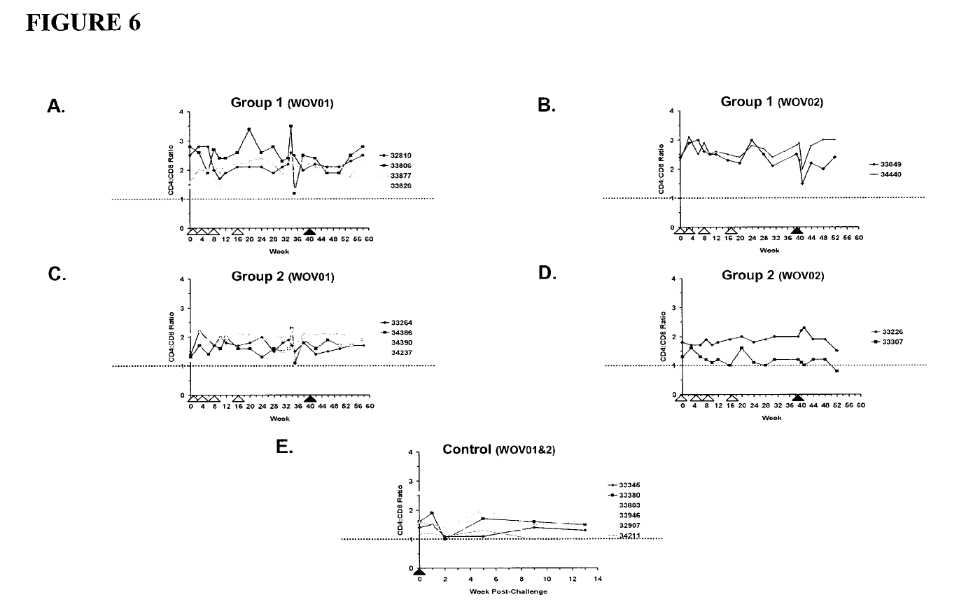

In both group 1 (FIG. 6A-B) and group 2 (FIG. 6C-D) vaccinated animals, CD4:CD8 levels remained relatively consistent, as expected, with no significant decline in CD4+ cells post-challenge. Similarly, unvaccinated control animals (FIG. 6E) remained relatively healthy post-challenge and maintained a stable CD4:CD8 ratio.

### Example 9

### Lymphocyte Proliferation

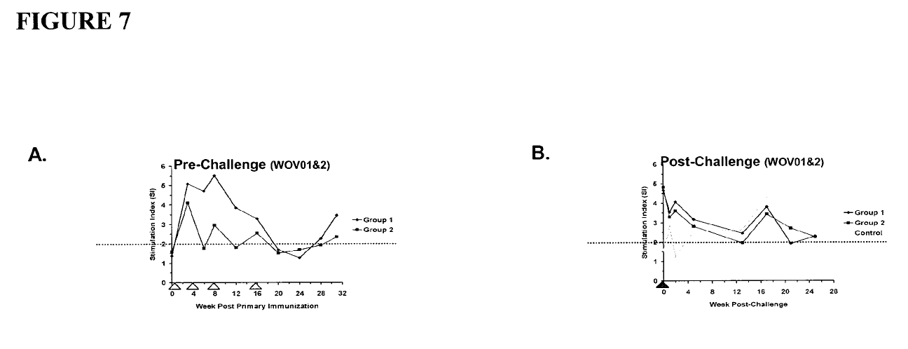

In order to determine whether or not T-cells had been primed for HIV-1 specific clonal expansion following vaccination, lymphocyte proliferation assays were performed. Samples were collected at various timepoints both pre- (FIG. 7A) and post-challenge (FIG. 7B) for both group 1 and 2 animals, as well as for post-challenge controls. Cells were stimulated by AT-2 inactivated HIV-1_{MN} virus for 6 days, and proliferation of CD4+ lymphocytes measured by incorporation of radio-labelled thymidine. A stimulation index (i.e. proliferation of stimulated vs. non-stimulated cells) of 2 was set as the cutoff value. As shown in FIG. 6A, both group 1 and group 2 animals showed a significant response to HIV-1 antigen during the vaccination phase. Group 1 animals, which received an initial inactivated-virus vaccination followed by 3 recombinant adenovirus boosts, showed a rapid and sustained proliferative response through 7/10 timepoints (70%). As well, group 2 animals, which received an initial recombinant adenovirus vaccination followed by 2 subsequent recombinant adenovirus and one final inactivated-virus boost, also showed strong proliferative responses through 4 of 10 timepoints (40%) and corresponded notably with vaccination timepoints at weeks 3, 8, and 16.

Post-challenge (FIG. 7B), both group 1 and group 2 animals showed an immediate and prolonged proliferative response to HIV-1 antigen. Both groups had positive stimulation indices through 7/8 (88%) timepoints, with group 1 vaccinated animals exhibiting a slightly stronger response. Control animals which showed no sign of HIV-1 specific proliferation prior to challenge, began showing some mild response to HIV-1 antigen 1 week after exposure to the SHIV challenge viruses. This response is most likely due to viral epitopes present in the challenge viruses themselves. Note that the apparently high level of HIV-1 specific T-cell stimulation observed in control animals at week 17 post-challenge correlates with animal TB testing, and should not be taken as indicative, as all control animals demonstrated an elevated CBC at this timepoint.

The results show that the vaccination approach taken was capable of inducing HIV-1 specific T-cell proliferative responses. Of the two protocols tested, group 1 animals appeared to show a stronger and more prolonged immune response relative to group 2 animals suggesting that inactivated-virus priming, followed by recombinant adenovirus boost, may be a more efficient method to induce a strong immune response.

### Example 10

### Cytotoxic T Lymphocyte (CTL) Response

One aspect of the immune response which is believed to be necessary to control HIV-1 infection is the development of CD8+ cytotoxic T lymphocytes (CTLs) as part of a cell-mediated immune response. To assess the CTL response in vaccinated animals, interferon-gamma (IFN-γ) ELISPOT assays were performed. A pool of 20 (15-mer) peptides, representing conserved epitopes of the HIV-1 Gag protein, were used to stimulate IFN-γ production by PBMCs isolated from group 1 and group 2 animals both pre- and post-challenge. PBMCs from an HIV-1 sero-positive donor served as the positive control for these experiments.

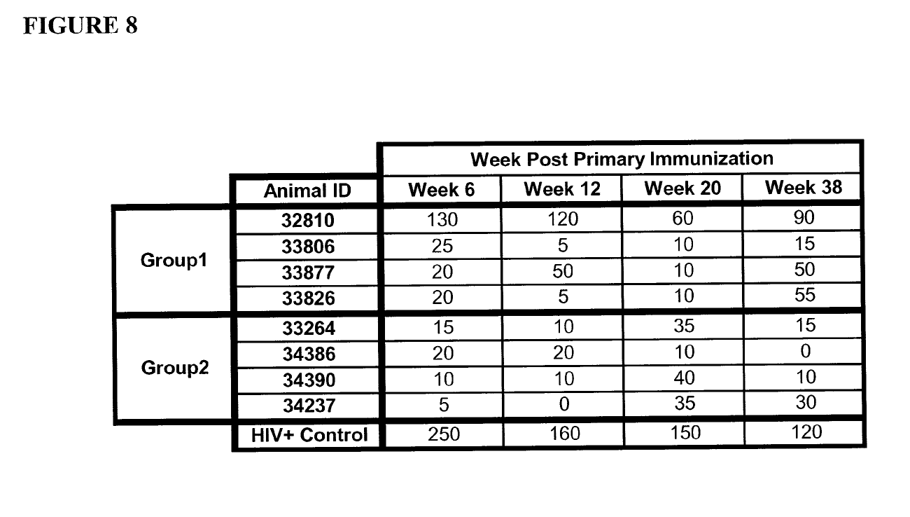

The results of the IFN-γ ELISPOT assays are summarized in FIG. 8.

While both groups of animals showed some response to the HIV-1 Gag peptide pool selected, most fell below the cutoff value of 50 IFN-γ secreting cells per million PBMCs. Only group 1 animals showed a sustained immune response, particularly following viral challenge (3/4 animals showed a positive ELISPOT response at week 38, 5 weeks post-challenge). This is consistent with the results of lymphoproliferative assays (FIG. 7), which showed a more robust response in the group 1 animals to HIV-1 specific antigen relative to group 2 animals.

This relatively weak response demonstrated by both groups 1 and 2 may be due in part to epitope selection. The pool of 20 HIV-1 Gag peptides selected may not have been sufficient to stimulate IFN-γ secretion from isolated PBMCs, or not specific to the regions targeted by the animals' immune response. As shown in FIG. 8, vaccinated animals from both group 1 and 2 cleared virus following challenge more efficiently than unvaccinated controls, suggesting indirectly the presence of an active CTL response.

### Example 11

### Plasma Viral Load Measurement

The SHIV challenge used in these experiments was a combination of two non-pathogenic strains, SHIV_{89.6} and SHIV_{SF162p4}. Due to their non-pathogenic nature, measurement of clinical disease progression would thus be insufficient to monitor any protective effects of vaccination. Instead, levels of viral RNA were measured by branched DNA (bDNA) assay, which determined the number of copies of the virus present per ml of plasma. This gives us an accurate measurement of the amount of virus present in the blood, down to a detection limit of Log 2.1 copies/ml.

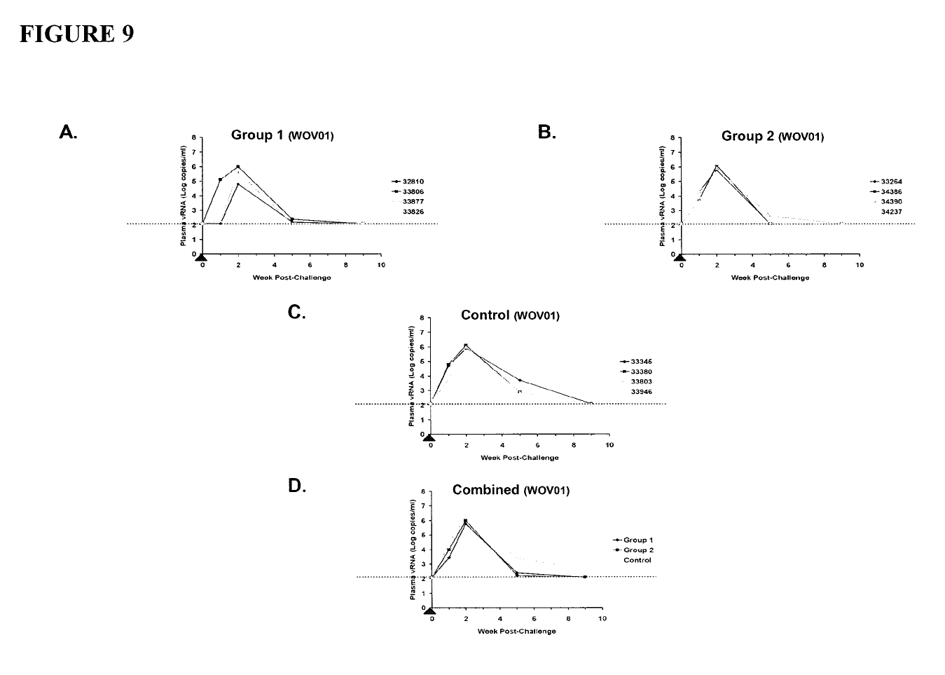

Group 1 and 2 animals showed a similar disease course (FIGS. 9A-B), with viral loads peaking at ˜10⁵-10⁶ copies/ml by two weeks post-challenge. Levels of plasma vRNA then decreased sharply to <10³ copies/ml by week 5 and below limits of detection by week 9. Control, unvaccinated animals also showed a peak viral load at week 2, with slightly elevated levels of 10⁶-10⁷ copies/ml (FIG. 9C). Further, viral loads declined more slowly than in vaccinated animals, with controls still exhibiting levels of 10³-10⁵ copies/ml at 5 weeks post-challenge, eventually tapering off by 9 weeks in most animals.

Overall, the combined data and comparison of all three groups (FIG. 9D), shows that vaccinated animals from groups 1 and 2 cleared virus significantly more rapidly than unvaccinated controls, as evidenced by reduced plasma vRNA levels at week 5 post-challenge.

### Example 12

### Antibody Response

The other important aspect of the immune response necessary to control HIV-1 infection is the development of a strong humoral, or antibody-mediated response. To assess the development and production of HIV-1 specific antibodies following vaccination and in response to viral challenge, serum samples were analyzed by enzyme-linked immunosorbent assay (ELISA). HIV-1 specific antibodies were detected using purified HIV-1_{IIIB} viral lysate as the capture antigen.

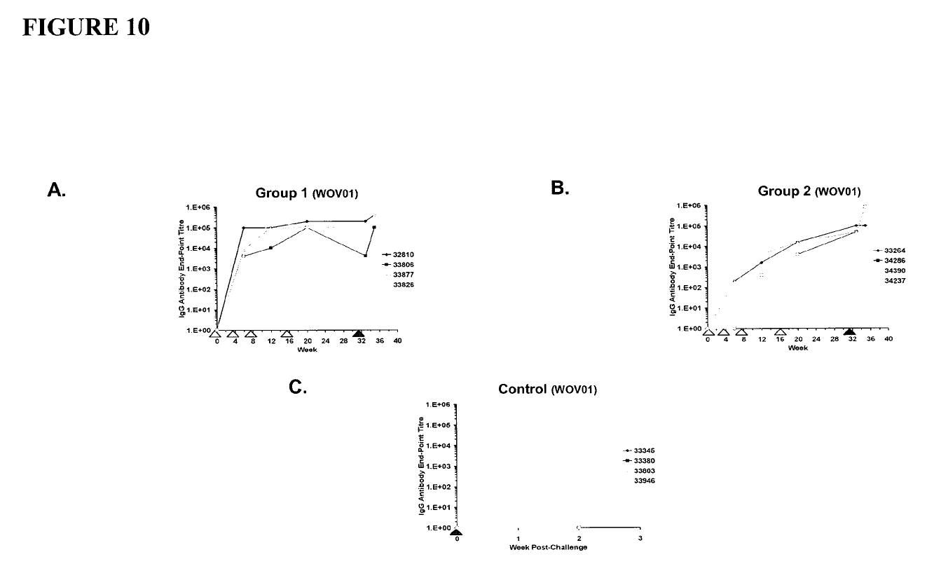

Animals in group 1 (FIG. 10A) rapidly developed a strong HIV-1 specific antibody response (10⁴-10⁵) following initial inactivated-virus prime and recombinant adenovirus boost. This response was further increased (>10⁵) by subsequent recombinant adenovirus boosts at weeks 8 and 16. Following SHIV challenge at week 33 the antibody response further increased to ˜10⁶. Group 2 animals (FIG. 10B) had a more delayed antibody development to 10³-10⁴ by 12 weeks. Inactivated-virus boosting at week 16 however, induced a significant and prolonged increase in antibody titres for several months. Again, following SHIV challenge at week 33 the antibody levels were elevated to ˜10⁶ indicating the presence of a memory response. Conversely, control animals showed no HIV-1 specific antibody production in response to the SHIV challenge (FIG. 10C).

These results show that vaccination is capable of inducing a strong and lasting antibody-mediated, humoral immune response. In particular, the protocol administered to group 1 animals (inactivated-virus prime followed by recombinant adenovirus boosting) elicited a rapid and robust response which persisted for several months and after challenge. This type of strong antibody response is an important factor in establishing protective immunity and may contribute to the ability of vaccinated animals to control and clear viral infection post-challenge discussed previously (see text and FIG. 9D).

### Example 13

### Conclusion

Our results strongly suggest that whole, AT-2 inactivated HIV-1 priming followed by two or three boost immunizations with recombinant adenoviruses carrying the HIV gag gene fused with either B- or T-cell epitopes elicit both humoral and cellular immune responses. This type of vaccination can be used to prevent HIV-1 infection as well as to treat HIV-1 infected individuals who are still immunocompetent. Human clinical trials with prime-boost vaccination are strongly recommended.

### REFERENCES

All publications and patents mentioned herein, including those references listed below, are hereby incorporated by reference in their entirety as if each individual publication or patent was specifically and individually incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

1. A method of preventing or treating a lentiviral infection comprising administering to a patient in need thereof, (a) an effective amount of a vaccine comprising a recombinant lentivirus having a glycoprotein 120 signal sequence, wherein said glycoprotein 120 signal sequence is selected from the group consisting of the polypeptide sequences listed as SEQ ID NO 3-6, or a functional fragment or variant thereof, wherein said functional fragment or variant thereof contains no more than one (1) positively charged amino acid and (b) an effective amount of a recombinant replication-defective adenovirus vector comprising a nucleic acid encoding a lentiviral protein.

2. The method of claim 1, wherein (a) is administered to the patient before (b) is administered to the patient.

3. The method of claim 2, wherein (b) is administered to the patient more than one time over the course of treating or preventing.

4. The method of claim 1, wherein (a) is administered to the patient in need thereof and (b) is administered to the patient in need thereof at about weeks three, eight and sixteen post-administration of (a).

5. The method of claim 1, wherein the lentiviral infection is a HIV infection.

6. The method of claim 5, wherein (a) is a recombinant human immunodeficiency virus-1 (HIV-1), wherein the natural signal sequence (NSS) of the HIV-1 envelope glycoprotein gp120 of the virus is replaced with a signal sequence selected from the group consisting of melittin signal sequence (MSS) and interleukin 3 signal sequence (ILSS).

7. The method of claim 6, wherein the effective amount of (a) is between about 0.10 mg/kg to about 0.23 mg/kg.

8. The method of claim 5, wherein (b) is a replication-defective recombinant adenoviral vector comprising a Ad5 genome having a deleted E1A gene region, wherein a HIV gene is inserted in said E1A region.

9. The method of claim 8, wherein the HIV gene is HIV-1 gag or HIV-2 gag.

10. The method of claim 8, wherein a HIV gene and at least one neutralizing or T-cell epitope is inserted in said E1A region.

11. The method of claim 10, wherein the HIV gene is HIV-1 gag or HIV-2 gag.

12. The method of claim 11, wherein the at least one neutralizing or T-cell epitope is selected from the group consisting of any of SEQ ID NOs: 14 through 34.

13. The method of claim 8, wherein the effective amount of (b) is between about 8.46×10⁸ mg/kg to about 2.21×10⁹ mg/kg.

14. A kit, comprising, (a) a dose of an effective amount of a vaccine comprising a recombinant lentivirus having a glycoprotein 120 signal sequence, wherein said glycoprotein 120 signal sequence is selected from the group consisting of the polypeptide sequences listed as SEQ ID NO 3-6, or a functional fragment or variant thereof, wherein said functional fragment or variant thereof contains no more than one (1) positively charged amino acid and (b) at least one dose of an effective amount of a recombinant replication-defective adenovirus vector comprising a nucleic acid encoding a lentiviral protein.

15. The kit of claim 14, wherein (a) and (b) are formulated in a pharmaceutically acceptable carrier.

16. The kit of claim 14, further comprising instructions for use.


SEQUENCE LISTING (35 sequences)

SEQ ID NO: 1 - protein, 31 aa, Artificial Sequence
  Description of Artificial Sequence Synthetic peptide
MRVKEKKTQHLWRWGWRWGTMLLGMLMICSA

SEQ ID NO: 2 - protein, 31 aa, Artificial Sequence
  Description of Artificial Sequence Synthetic peptide
MXVXEXKTQHLWXWGWXWGTMLLGMLMICSA

SEQ ID NO: 3 - protein, 31 aa, Artificial Sequence
  Description of Artificial Sequence Synthetic peptide
MRVAEIKTQHLWRWGWRWGTMLLGMLMICSA

SEQ ID NO: 4 - protein, 31 aa, Artificial Sequence
  Description of Artificial Sequence Synthetic peptide
MIVKEKKTQHLWIWGWIWGTMLLGMLMICSA

SEQ ID NO: 5 - protein, 31 aa, Artificial Sequence
  Description of Artificial Sequence Synthetic peptide
MRVVEIKTQHLWIWGWIWGTMLLGMLMICSA

SEQ ID NO: 6 - protein, 31 aa, Artificial Sequence
  Description of Artificial Sequence Synthetic peptide
MIVAEIKTQHLWIWGWIWGTMLLGMLMICSA

SEQ ID NO: 7 - protein, 26 aa, Artificial Sequence
  Description of Artificial Sequence Synthetic peptide
MKFLVNVALVFMVVYISYIYADPINM

SEQ ID NO: 8 - protein, 25 aa, Artificial Sequence
  Description of Artificial Sequence Synthetic peptide
MLLLLLMLFHLGLQASISGRDPINM

SEQ ID NO: 9 - protein, 28 aa, Human immunodeficiency virus type 1
MRVKEKYQHLWRWGWKWGTMLLGILMIC

SEQ ID NO: 10 - protein, 26 aa, Human immunodeficiency virus type 1
MRVKEIRKNWQHLRGGILLLGMLMIC

SEQ ID NO: 11 - protein, 27 aa, Human immunodeficiency virus type 1
MRVRETKRNYQHLWKWGTMLLGMLMIC

SEQ ID NO: 12 - protein, 28 aa, Human immunodeficiency virus type 1
MRVKETQMNWPNLWKWGTTLILGLVIIC

SEQ ID NO: 13 - protein, 21 aa, Human immunodeficiency virus type 1
MKFLVNVALVFMVVYISYIYA

SEQ ID NO: 14 - protein, 15 aa, Human immunodeficiency virus type 1
SIPIGPGRAFYATGD

SEQ ID NO: 15 - protein, 15 aa, Human immunodeficiency virus type 1
SVRIGPGQTFYATGA

SEQ ID NO: 16 - protein, 15 aa, Human immunodeficiency virus type 1
SIRIGPGQVFYRTGD

SEQ ID NO: 17 - protein, 15 aa, Human immunodeficiency virus type 1
SVRIGPGQTFYATGD

SEQ ID NO: 18 - protein, 15 aa, Human immunodeficiency virus type 1
RTPIGLGQALYTTRD

SEQ ID NO: 19 - protein, 15 aa, Human immunodeficiency virus type 1
RISLGPGRVFYTAGE

SEQ ID NO: 20 - protein, 15 aa, Human immunodeficiency virus type 1
SINLGPGQAIYATGA

SEQ ID NO: 21 - protein, 41 aa, Human immunodeficiency virus type 1
PCRIKQIVNMWQEVGKAMYAPPISGQIRCSSNITGLLLTRD

SEQ ID NO: 22 - protein, 20 aa, Human immunodeficiency virus type 1
RAMYAPPIAGNITCKSNIRS

SEQ ID NO: 23 - protein, 13 aa, Human immunodeficiency virus type 1
QAMYAPPISGKIN

SEQ ID NO: 24 - protein, 6 aa, Human immunodeficiency virus type 1
ELDKWA

SEQ ID NO: 25 - protein, 9 aa, Human immunodeficiency virus type 1
IPRRIRQGL

SEQ ID NO: 26 - protein, 9 aa, Human immunodeficiency virus type 1
KEKGGLDGL

SEQ ID NO: 27 - protein, 10 aa, Human immunodeficiency virus type 1
TPGPGVRYPL

SEQ ID NO: 28 - protein, 10 aa, Human immunodeficiency virus type 1
ERYLKDQQLL

SEQ ID NO: 29 - protein, 17 aa, Human immunodeficiency virus type 1
AAENLWVTVYYGVPVWK

SEQ ID NO: 30 - protein, 9 aa, Human immunodeficiency virus type 1
ILKEPVHGV

SEQ ID NO: 31 - protein, 20 aa, Human immunodeficiency virus type 1
FRKQNPDIVIYQYMDDLYVG

SEQ ID NO: 32 - protein, 9 aa, Human immunodeficiency virus type 1
AIFQSSMTK

SEQ ID NO: 33 - protein, 10 aa, Human immunodeficiency virus type 1
EPVDPRLEPW

SEQ ID NO: 34 - protein, 9 aa, Human immunodeficiency virus type 1
PAEPVPLQL

SEQ ID NO: 35 - DNA, 24 bp, Artificial Sequence
  Description of Artificial Sequence Synthetic oligonucleotide
tcgtcgttttgtcgttttgtcgtt